(19) United States
(12) Patent Application Publication
(10) Pub. No.: US 2004/0241194 A1
(43) Pub. Date: Dec. 2, 2004

(54) Food supplement for animals

(75) Inventors: Thierry Picaud, Saint Gal Sur Sioule (FR); Amaury Greuter, Riom (FR)

Correspondence Address: HESLIN ROTHENBERG FARLEY & MESITI PC, 5 COLUMBIA CIRCLE, ALBANY, NY, 12203, US

(73) Assignee: Phytosynthese, Gannat (FR)

(21) Appl. No.: 10/872,102

(22) Filed: Jun. 18, 2004

Related U.S. Application Data
(63) Continuation of application No. PCT/FR02/04307, filed on Dec. 12, 2002.

(30) Foreign Application Priority Data
Dec. 26, 2001 (FR) 01.16886

(51) Int. Cl. A61K 39/00

(52) U.S. Cl. 424/265.1

(57) ABSTRACT

A food supplement for animals, comprises a mixture of a eucalyptus extract, a fenugreek extract, and a boldo extract.

The invention relates to a supplementary feedstuff which is intended for animals, in particular ruminants, poultry, rabbits and pigs. The invention also relates to the use of said alimentary supplement for manufacturing a composition which is intended for the curative or preventive treatment of ruminant, poultry, rabbit and pig coccidiosis.

Coccidiosis is a parasitic disease which affects animals, in particular the abovementioned animals, and which can develop suddenly or evolve over a period of several weeks. The parasite in question is termed Coccidia or Eimeria, and the species which are pathogenic or nonpathogenic differ depending on the animal. The developmental cycle of the parasite is well known and particularly complex and essentially takes place partly in the external environment and partly in the intestine of the infected animal. The symptoms which are observed correspond to a digestive syndrome with enteritis, with this frequently being hemorrhagic. In addition, acute coccidiosis can result, in certain cases, in the speedy death of the animal.

A large number of treatments have been suggested for treating this disease.

Thus, a first type of treatment consists, for example, in administering antibiotics. The document FR-A-2 356 667 describes, for example, preparing an antibiotic for treating and preventing poultry coccidiosis, this antibiotic being prepared from Streptomyces. The antibiotic described in the document WO-93/12800 is an antibiotic which is based on polycyclic acid ester and which is obtained by fermenting a microorganism called Actinomadura. Other antibiotics have also been described, for example in the documents EP-A-15 110, EP-A-63 238 and EP-A-255 335.

Another treatment consists in potentiating the immune functions of the animal by administering an immuno-potentiating and anti-infective agent which is obtained by mixing several bacilli. A method of this type is described, for example, in the documents WO-94/22 459 and WO-99/43 348.

Some documents also describe treatments involving the administration of synthesis molecules such as, for example, purines (EP-A-76 127), pyrroles (WO-01/34 632), riboflavin derivatives (FR-A-2 368 956), etc.

Document EP-A-47 662 describes another type of treatment which consists of a vaccine which is obtained from an attenuated strain of Eimeria necatrix.

Other treatments consist in administering, to the animal, a composition which is based on plants. Thus, document HU-A-96 02190, for example, describes an alimentary supplement for treating and preventing chicken coccidiosis which principally comprises garlic. Document EP-A-0 835 120 describes a pharmaceutical composition which is used, in particular, for treating poultry coccidiosis and which comprises a combination of thymol and carvacrol, which is obtained from oregano essential oil.

Document JP 07046963 describes an alimentary composition which is intended for preventing and treating coccidiosis and which contains terpenes such as perrillaldehyde, citral and borneol, or terpenes which are derived from eucalyptus oil or clove oil.

Document GB-A-2 321 583 describes a feedstuff which is to be chewed or licked by the animal and which is based on a mixture of molasses and calcium, with it also being possible for the mixture to comprise herbs such as, in particular, fenugreek.

Document FR-A-2 582 485 describes a composition for supplementing and equilibrating the animal diet and which is based on fish, yeast, vegetable oil and a phytotherapeutic complex of plants among which boldo is mentioned, in particular.

The Applicant discovered, entirely surprisingly, that combining eucalyptus, boldo and fenugreek made it possible to treat poultry, ruminant (calves, lambs, heifers and horses), rabbit and pig coccidiosis, both preventively and curatively, more efficiently than when using eucalyptus oil on its own.

Consequently, the invention relates to an alimentary supplement which is intended for animals and which is characterized in that it comprises, as a mixture, a eucalyptus extract, a fenugreek extract and a boldo extract.

In one advantageous embodiment, the eucalyptus extract belongs to the “globulus” variety.

In practice, the extract of eucalyptus leaves is present in the form of an essential oil which represents between 8 and 15% by weight of the supplement, advantageously between 9 and 13%.

The eucalyptus essential oil is obtained, in a known manner, by steam distilling Eucalyptus globulus leaves.

The fenugreek extract is obtained from the seed and is present in the form of a powder which in practice represents between 10 and 30% by weight of the alimentary supplement, preferably between 12 and 20%.

Fenugreek belongs to the family of leguminous plants and produces yellow seeds which contain an essential oil which has a very bitter, marked odor and which is known by the name triogonelline. In addition, the seed is very rich in lecithin, nucleoalbumin and iron in very rapidly assimilable form. In addition, fenugreek has the advantage of being included on the list of G.R.A.S. (Generally Recognized As Safe) plants in the American Code of Federal Regulations.

In order to restrict the malabsorption phenomena which frequently occur in coccidiosis, said alimentary supplement also contains a boldo leaf extract, which is employed because of its positive effect on digestion and which in practice represents from 10 to 20%, advantageously between 11 and 17%, by weight of the alimentary supplement.

The boldo leaves originate from a small tree which is termed “Pneumus boldus” and whose leaves contain a low quantity of boldine. The leaves are ground in order to form a yellow-green colored powder having accentuated aromatic fragrances and flavors.

In order to top up the composition to 100% by weight, the alimentary supplement additionally comprises a pharmaceutically acceptable excipient, in particular a powder derived from middlings.

As will be understood, the alimentary supplement of the invention can be integrated into the feed of the animal at the rate in practice of from 1 to 6 kg per tonne of complete feed, that is:

in the case of poultry, pigs and rabbits: from 6 to 12 g/100 kg of live weight (LW),

in the case of ruminants: from 4 to 6 g/100 kg of LW.

As already mentioned, the Applicant observed, entirely surprisingly, that the alimentary supplement of the invention was very effective in treating coccidiosis in some animals.

Consequently, the invention also relates to the use of the alimentary supplement, as previously described, for manufacturing a composition which is intended for the preventive or curative treatment of coccidiosis in ruminants, poultry, rabbits and pigs.

When the alimentary supplement is used, in this treatment, for the purpose of obtaining a preventive effect, the quantity of eucalyptus essential oil which is administered is from 0.7 to 0.15 g/100 kg of LW in the case of poultry, pigs and rabbits and from 0.5 to 0.7 g/100 kg of LW in the case of ruminants.

On the other hand, when the alimentary supplement is used for the purpose of curative treatment, the quantity of essential oil which is administered to the animal is between 0.1 and 0.3 g/100 kg of LW in the case of poultry, pigs and rabbits and from 0.7 to 0.15 g/100 kg of LW in the case of ruminants.

The invention, and the advantages which ensue from it, will emerge more clearly from the following implementation examples, which support the appended figures.

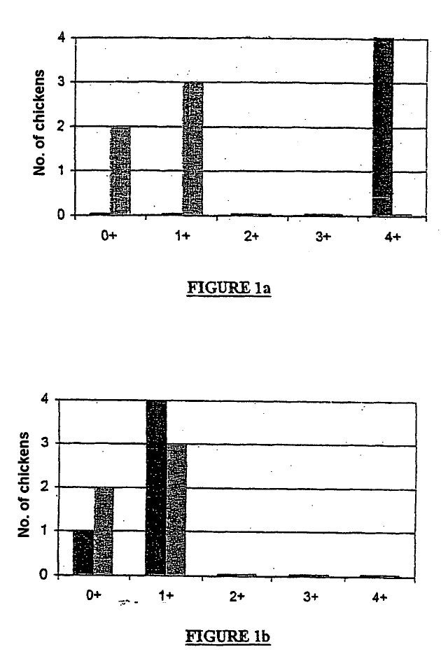

FIG. 1 depicts oocyst excretion in the duodenum (1a) or the caecum (1b) before and after treating with the composition of the invention.

### EXAMPLE 1

### Preparing the Composition of the Invention

An alimentary supplement is prepared having the following composition (Please verify whether this formula corresponds to that of EIMERICOX):

eucalyptus essential oil: 15%

boldo extract: 12%

fenugreek seed: 15%

middlings qs: 100%

The essential oil is first of all absorbed on middlings. (production of a premix). The other ingredients are then added.

### EXAMPLE 2

a/ Efficacy in Lambs

The objective is to determine the anticoccidial activity of the phytotherapeutic product of the invention (product A) in a treatment directed against coccidial infestation in housed lambs.

### Material and Method

1—Experimental Animals

The experimental animals are 77 crossbreed lambs (crossbreed×charolais ewes) born from the end of October to the middle of November on the same farm. They are reared with their mothers in a sheep barn, being suckled for three months and then weaned and fattened.

2—Treatment

Two batches of lambs are assembled, 10 days after birth, on the basis of weight, sex and the number of individuals per ewe.

Control batch: 34 lambs are kept without any anticoccidial treatment until sale (12 single lambs, 22 twin lambs (17 males and 17 females)).

Invention batch: 33 lambs are given product A from approximately 10 days after birth until sale, at a dosage of 6 g/100 kg of live weight/day, that is 2 kg/tonne of feed. The phytotherapeutic product is incorporated into the feed, which is provided ad lib to the lambs (13 single lambs, 20 twin lambs (15 males and 18 females)).

3—Monitoring

Parasite Monitoring

The coccidial excretion was quantified by means of individual coproscopic examinations (Calamel-Soulè technique) carried out on dropping samples obtained from 15 lambs from each batch (the same lambs at each control). The examinations are carried out on D0, D21 and D45. The coccidial species are identified in the mixture of the faeces from each batch.

The anticoccidial efficacy is calculated from the geometric mean, in accordance with the following principle:

$$\frac{\begin{matrix}Number of o . p . g . on Dx , control batch - \\ number of o . p . g . on Dx , batch A\end{matrix}}{Number of o . p . g . on Dx , control batch}$$

### RESULTS

1—Anticoccidial Activity

On Day 0, the excretion is 306.2 coccidial oocysts per gram (o.p.g.) in the case of the control batch and 418 o.p.g. in the case of batch A. In the case of the control batch, an excretion of 18 146 o.p.g. is recorded on D21, while that recorded on D45 is 1418 (arithmetic mean) which relates to all the lambs in the batch. Product A exhibits anticoccidial activity. The antiparasitic efficacy is 99.6% on D21 and 96.1% on D45, based on the reduction in excretion (table I). This anticoccidial activity reduced the number of This anticoccidial activity reduced the number of excreting lambs. The efficacy is 60% on D21 and 30% on D45 (table II).

TABLE 1
Anticoccidial activity of the composition of
the invention

| | | Number of coccidial oocysts | | |
|---|---|---|---|---|
| | | Nov. 21, 2000 | Dec. 12, 2000 | Jan. 03, 2001 |
| | | D0 | D21 | D45 |
| Control | (1) | 3062.0 | 18 146.0 | 1418.0 |
| batch | (2) | 12.4 | 6509.5 | 373.2 |
| Batch A | (1) | 418.0 | 8206.0 | 64.0 |
| | (2) | 20.9 | 23.9 | 14.5 |
| Efficacy | (*) | | 99.6% | 96.1% |

(1) Arithmetic mean
(2) Geometric mean
(*) Calculation performed on the geometric mean

TABLE II
Anticoccidial activity of the composition of
the invention

| | Number of positive lambs | | | | |
|---|---|---|---|---|---|
| | Nov. 21, 2000 | Dec. 12, 2000 | Jan. 03, 2001 | Efficacy (%) | |
| | D0 | D21 | D45 | D21 | D45 |
| Control | 4/10 | 10/10 | 9/10 | | |
| batch | | | | | |
| Batch A | 5/10 | 4/10 | 6/10 | 60 | 30 |

In the case of the control batch, the overall results on the growth of the lambs show a weight gain of 14.7 kg and a mean rearing period of 105.9 days. In the case of batch A, the weight gain is 15.4 kg for a mean rearing period of 85 days. The increase in growth is 4.8%, and the reduction in the rearing period is 23.3%, as compared with the control batch (table III).

TABLE III
Animal husbandry balance sheet after
monitoring the coccidias when using batch A

| | Weight (kg) | | | | |
|---|---|---|---|---|---|
| | Nov. 21, | Dec. 12, | Jan. 03, | Weight | Rearing |
| | 2000 | 2000 | 2001 | gain | period |
| | D0 | D21 | D45 | (kg) | (days) |
| Control | 9.2 ± 2.6 | 14.8 ± 3.7 | 23.9 ± 5.2 | 14.7 ± 4.1 | 105.9 ± 30.8 |
| batch | | | | 139 g/d | |
| Batch A | 9.2 ± 1.8 | 15.1 ± 2.8 | 24.2 ± 4.3 | 15.4 ± 2.8 | 85.9 ± 23.8 |
| | | | | 179 g/d | |

| | | |
|---|---|---|
| Difference | +4.8% | −23.3% |

In the case of the replacement ewe lambs, an improvement of 8.8% in weight gain is recorded in the case of batch A as compared with the control batch (table IV). This tendency is observed in the lambs in batch A which are destined for slaughter, with a growth greater than 1.3% and a rearing period which is reduced by 13.2% (table V).

TABLE IV
Weight gain in the ewe lambs

| | Weight (kg) | | | Weight | |
|---|---|---|---|---|---|
| | Nov. 21, | Dec. 12, | | gain | Rearing |
| | 2000 | 2000 | Jan. 03, 2001 | (kg) | period |
| | D0 | D21 | D45 | D0-D45 | (days) |
| Control | 9.6 ± 3.4 | 15.2 ± 4.4 | 23.3 ± 4.3 | 13.6 ± 4.9 | 65.4 ± 3.4 |
| batch | | | | 208 g/d | |
| Batch A | 8.8 ± 1.6 | 15.0 ± 2.9 | 23.7 ± 4.0 | 14.8 ± 2.6 | 66.0 ± 4.8 |
| | | | | 224 g/d | |

| | |
|---|---|
| Difference | +8.8% |

TABLE V
Weight gain in the housed lambs

| | Weight (kg) | | | Weight | |
|---|---|---|---|---|---|
| | Nov. 21, | Dec. 12, | Jan. 03, | gain | Rearing |
| | 2000 | 2000 | 2001 | (kg) | period |
| | D0 | D21 | D45 | D0-Sale | (days) |
| Control | 9.0 ± 2.2 | 14.7 ± 3.5 | 24.1 ± 5.7 | 15.15 ± 3.8 | 123.2 ± 17.8 |
| batch | | | | 123 g/d | |
| Batch A | 9.7 ± 2.1 | 15.2 ± 2.8 | 24.9 ± 4.7 | 15.3 ± 3.2 | 108.8 ± 13.5 |
| | | | | 140 g/d | |

| | | |
|---|---|---|
| Difference | +1.3% | −13.2% |

b/ Efficacy in Poultry

1. Anticoccidial Activity

The anticoccidial activity of the composition of the invention was assessed on 22 000 chickens suffering from coccidiosis, with the treatment being carried out over 3 days at a rate of 1 ml/l.

FIGS. 1a and 1b depict the duodenal (FIG. 1a) and caecal (FIG. 1b) oocyst excretions.

As these histograms show, whatever the level of oocyst excretion before treatment (left-hand bars), a net reduction in excretion is seen, both in the duodenum and in the caecum, after 3 days of treatment (right-hand bars).

2. Influence on Growth

a/ Open-Label Study

The present study demonstrates the efficacy of the composition of the invention, as compared with a control chemical composition (monensin), on the growth of two batches of chickens.

The parameters considered are the performance index and the mean daily weight gain. The composition of the invention is administered at the rate of 1.5 kg per tonne.

The results are reproduced in tables VI and VII below.

TABLE VI

| | | | Mean | | | | |
|---|---|---|---|---|---|---|---|
| | Number | | daily | | Mean | | |
| | of | Age in | weight | | weight | | Performance |
| | animals | days | gain in g | Error | in kg | Error | index |
| Control | 98 300 | 82.84 | 25.32 | | 2.083 | | 87 |
| Composition of | 38 229 | 82.36 | 26.36 | ÷1.04 | 2.171 | +88 g | 89 |
| the invention | | | | | | | |

TABLE VII

| | | | Mean | | | | |
|---|---|---|---|---|---|---|---|
| | Number | | daily | | Mean | | |
| | of | Age in | weight | | weight | | Performance |
| | animals | days | gain in g | Error | in kg | Error | index |
| Control | 30 129 | 81.98 | 26.46 | | 2.169 | | 95 |
| Composition of | 12 762 | 81.66 | 27.55 | +1.09 | 2.250 | +81 g | 97 |
| the invention | | | | | | | |

As this study demonstrates, the mean daily weight gain in grams for a chicken treated with the composition of the invention is greater than that achieved using the control chemical coccidiostat. The performance index resulting from combining the different factors, in particular mean daily weight gain, feed conversion index and chicken per m², is also improved.

b/ Study Performed on White Free-Range Chickens

This test is conducted under the same conditions by administering 2 kg of the composition of the invention per tonne to four batches of chickens.

The results are given in table VIII below.

TABLE VIII

| | | | | | | | | Feed conversion | |
|---|---|---|---|---|---|---|---|---|---|
| | | | | Mean | | | | index (quantity | |
| | | Number | Age | daily | | Mean | | of feed for | |
| | | of | in | weight | | weight | | obtaining 1 kg | |
| | | animals | days | gain in g | Error | in kg | Error | of chicken) | Error |
| General | Control | 161 796 | 83.12 | 25.46 | | 2.116 | | 2.979 | |
| Mean | Composition | 172 553 | 82.84 | 25.74 | 0.28 | 2.132 | 0.016 | 2.925 | −0.054 |
| | of the | | | | | | | | |
| | invention | | | | | | | | |
| Mean of | Control | 51 684 | 82.32 | 26.30 | | 2.265 | | 2.810 | |
| the 1^{st} | Composition | 58 651 | 82.43 | 26.28 | −0.02 | 2.166 | 0.001 | 2.763 | −0.027 |
| third | of the | | | | | | | | |
| | invention | | | | | | | | |
| Mean of | Control | 55 584 | 83.64 | 25.48 | | 2.131 | | 2.960 | |
| the 2^{nd} | Composition | 55 634 | 83.40 | 25.96 | 0.48 | 2.165 | 0.034 | 2.917 | −0.043 |
| third | of the | | | | | | | | |
| | invention | | | | | | | | |
| Mean of | Control | 54 528 | 83.37 | 24.64 | | 2.054 | | 3.167 | |
| the 3^{rd} | Composition | 58 268 | 28.72 | 24.98 | 0.34 | 2.066 | 0.012 | 3.083 | −0.084 |
| third | of the | | | | | | | | |
| | invention | | | | | | | | |

As this table shows, both the mean daily gain in weight and the feed conversion index (FCI) are improved in the case of the composition of the invention.

### EXAMPLE 3

### Synergistic Effect of Eucalyptus, Fenugreek and Boldo

This example aims to demonstrate the improvement in the efficacy of treating coccidiosis which is obtained by the mixture of the invention as compared with a simple eucalyptus extract.

a/ Assessing the Nitrate Level 6 Days After Infection

The nitrate measurement points to the reactivity of the macrophages. It indirectly characterizes the immune reaction, and therefore defense, of the animals. The lower the nitrate level, the less substantial is the inflammatory reaction. Each of the birds is infected with 1000 E. acervulina oocysts and 1000 E. tenella oocysts.

The following compositions are tested:

A: eucalyptus essential oil

B: composition of the invention (Example 1)

U: no treatment

V: no infection

The results are given in the following table:

| Composition | No. of birds | NO⁻₂ + NO⁻₃ level (μM) |
|---|---|---|
| A | 8 | 14.60 |
| B | 8 | 18.87 |
| U | 8 | 14.47 |
| V | 8 | 7.40 |

As is evident from this table, the composition of the invention brings about an improvement as compared with eucalyptus on its own.

b/ Excretion of Oocysts After 5 to 8 Days of Infection

The birds are infected with high doses of oocysts (10 000 E. acervulena+10 000 E. tenella). The number of oocysts excreted after 5 to 8 days of infection is measured.

The results are shown in the following table:

| Composition | No. of birds | NO⁻₂ + NO⁻₃ level (μM) |
|---|---|---|
| A | 8 | 2.22 |
| B | 8 | 0.93 |
| U | 8 | 2.31 |

As is evident from this table,. the composition of the invention has a particularly strong effect on the excretion of the oocysts.

1. An alimentary supplement which is intended for animals, comprising a mixture of:
a eucalyptus extract,
a fenugreek extract,
a boldo extract,
and, where appropriate, a pharmaceutically acceptable excipient.

2. The alimentary supplement as claimed in claim 1, wherein the eucalyptus extract is present in the form of an essential oil which represents between 8 and 15% by weight of the supplement.

3. The alimentary supplement as claimed in either of the preceding claims, wherein the fenugreek extract comprises fenugreek seed extract and the fenugreek seed extract is present in the form of a powder.

4. The alimentary supplement as claimed in claim 3, wherein the fenugreek seed extract represents between 10 and 30% by weight of the alimentary supplement.

5. The alimentary supplement as claimed in claim 1, wherein the boldo extract represents from 10 to 20% by weight of the alimentary supplement.

6. The use of the alimentary supplement as claimed in one of claims 1 to 5 for manufacturing a composition which is intended for the preventive or curative treatment of ruminant, poultry, rabbit and pig coccidiosis.

7. The use of the alimentary supplement as claimed in one of claims 1-5 for preventative treatment of ruminant, poultry, rabbit or pig occidiosis.

8. The use of the alimentary supplement as claimed in one of claims 1-5 for curative treatment of ruminant, poultry, rabbit or pig occidiosis.
